(19) United States
(12) Patent Application Publication
(10) Pub. No.: US 2016/0166722 A1
(43) Pub. Date: Jun. 16, 2016

(54) Scented Lenticular Air Freshener

(71) Applicant: David Mudrick, Prescott, AZ (US)

(72) Inventor: David Mudrick, Prescott, AZ (US)

(73) Assignee: ORION PHOTO INDUSTRIES, INC., Prescott, AZ (US)

(21) Appl. No.: 14/568,521

(22) Filed: Dec. 12, 2014

(51) Int. Cl. A61L 9/12; G02B 27/22

(52) U.S. Cl. CPC A61L 9/12; G02B 27/2214

(57) ABSTRACT

The present invention describes an improved scented lenticular air freshener. In a preferred embodiment, a scent cartridge may be inserted in a separate scent sleeve which may be affixed to a lenticular image. Such a scent sleeve may also include a plurality of perforations to facilitate dispersal of a scent into the environment. This scent sleeve may act as a physical barrier to prevent, for example scented oils from penetrating the lenticular image and destroying the corresponding lenticular image and/or lenticular lenses.

### TECHNICAL FIELD

Generally, the inventive technology disclosed herein relates to techniques, systems, methods and apparatus for providing a scent delivery system. Such scent delivery system may be incorporated into casings for souvenirs and other similar items. In a preferred embodiment, such a scent delivery system may be incorporated into casings for lenticular devices, or devices that display a lenticular image. Additional embodiments of the inventive technology may include scent personalization as well as replaceable scent elements. Each of these embodiments will be discussed generally below.

### BACKGROUND OF THE INVENTION

Traditional scent delivery systems incorporate scented oils or perfumes impregnated into a adsorbent material. Once exposed, the scented oils begin to evaporate and are dispersed into the air at a generally controlled rate. However, such traditional impregnation based systems are impractical or impossible for a variety of items, such as plastics or other non-adsorbent material based products. In one example, lenticular images, because of their generally flat presentation and visually dynamic eye-catching characteristics may be particularly suited for scent impregnation. However, traditional scent impregnation methods fail in this regard as the scented oils used for traditional scent impregnation are incompatible with, and destroy the lenticular printing inks as well as destroy the plastic lenses necessary to achieve the desired dynamic image switching. Such negative effects are exacerbated when an impregnated lenticular image product is sealed, for example in a plastic bag or container where the internal humidity is near 100%.

The foregoing problems regarding conventional scent delivery systems that are compatible with lenticular images and the like and processes may represent a long-felt need for an effective solution to the same. While implementing elements may have been available, actual attempts to meet this need may have been lacking to some degree. This may have been due to a failure of those having ordinary skill in the art to fully appreciate or understand the nature of the problems and challenges involved. As a result of this lack of understanding, attempts to meet these long-felt needs may have failed to effectively solve one or more of the problems or challenges here identified. These attempts may even have led away from the technical directions taken by the present inventive technology and may even result in the achievements of the present inventive technology being considered to some degree an unexpected result of the approach taken by some in the field.

### BRIEF DESCRIPTION OF THE DRAWINGS

FIG. 1: is a front view of a lenticular image showing a first image in one embodiment thereof;

FIG. 2A: is a back view of a lenticular image having an affixed scent sleeve with a plurality of perforations in one embodiment thereof;

FIG. 2B: is a scent cartridge in one embodiment thereof;

FIG. 3: is a back view of a lenticular image having an affixed scent sleeve with a plurality of perforations holding a scent cartridge in one embodiment thereof;

FIG. 4: is a top view of a lenticular image with a blow-up view of a plurality of lenticular lenses in one embodiment thereof;

FIG. 5: is a bottom view of a lenticular image in one embodiment thereof;

FIG. 6: is a side view of a lenticular image in one embodiment thereof;

FIG. 7: is a side view of a lenticular image in one embodiment thereof;

FIG. 8: is a front perspective view of a lenticular image showing a second image in one embodiment thereof.

### DETAILED DESCRIPTION OF THE INVENTION

The present invention includes a variety of aspects, which may be combined in different ways. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

In one embodiment, a scent sleeve (1) may be adapted to a souvenir, such as a lenticular image (2). In a preferred embodiment, a plurality of flat and/or static personalized images may be selected. These static images may then be interlaced together such that they may be flattened into individual, different frame files, and then digitally combined into a single final image. From there the interlaced image may be printed directly to the back of a piece of plastic having a plurality of thin lenses (5) molded into the opposite side. In an alternative embodiments, this interlaced image may be printed on paper, which is then bonded to, for example a plastic lens (2). In a preferred embodiment, the interlaced image may be printed to a substrate such as a synthetic paper and then laminated to the lens. Each lens on, in this case the plastic lens lenticular sheet may magnify a small portion of the interlaced image beneath it. As such, when the viewing angle of the lens changes, a different portion of the image is magnified. It should be noted that various lenticular effects are contemplated in the inventive technology. For example, products exhibiting lenticular transforming prints, lenticular animated prints, 3-D lenticular effects and lenticular stereoscopic effects among others.

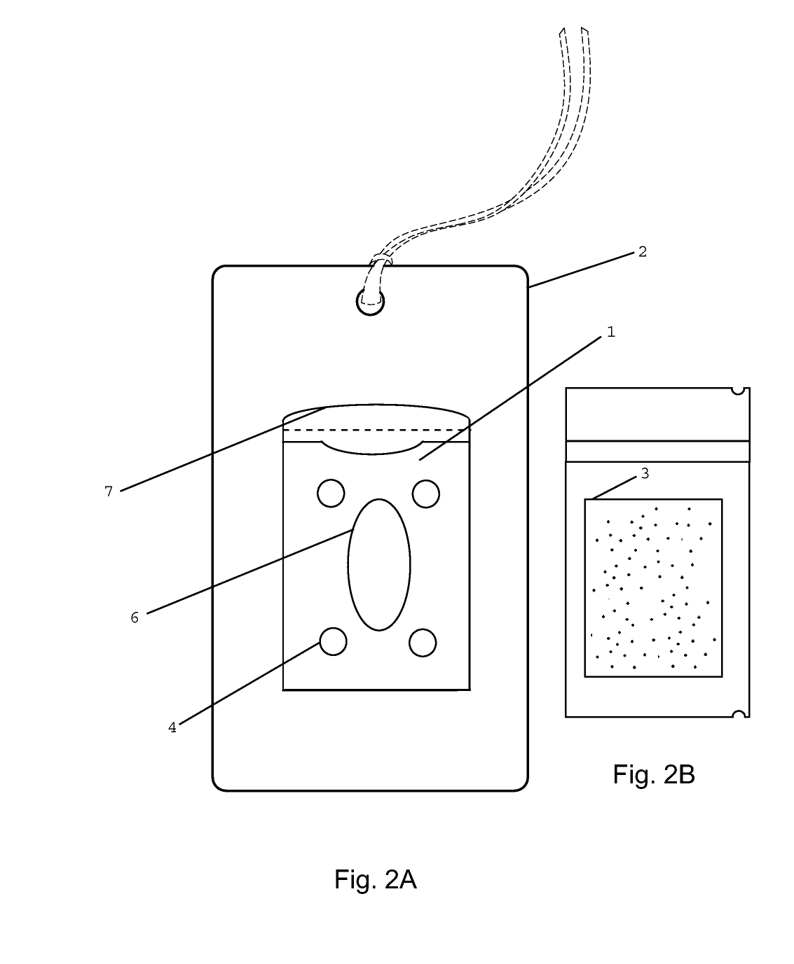

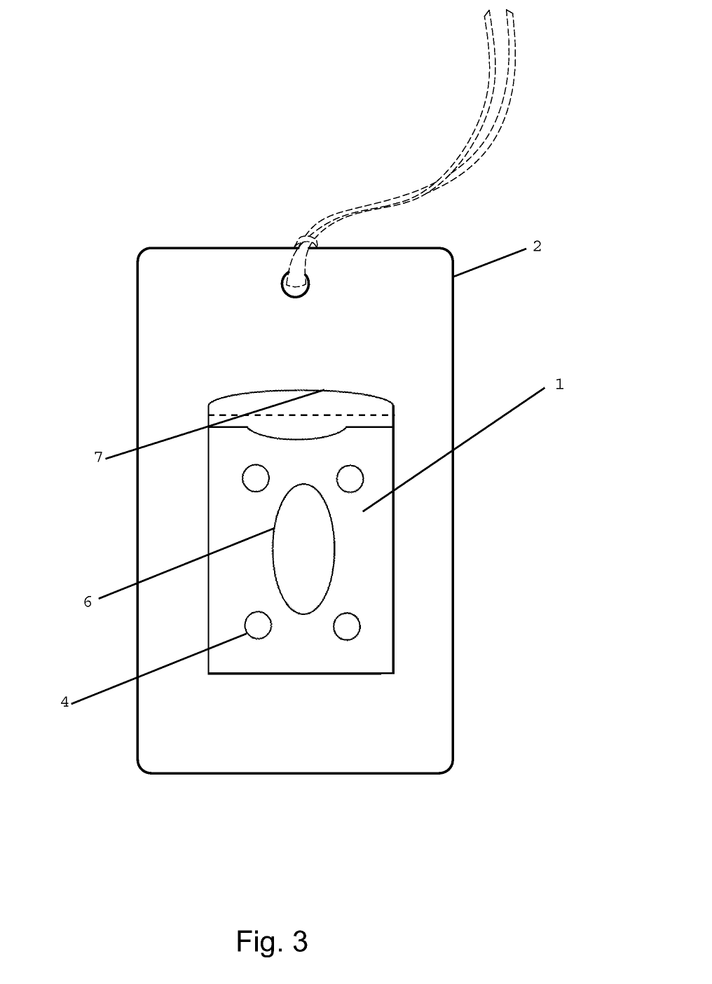

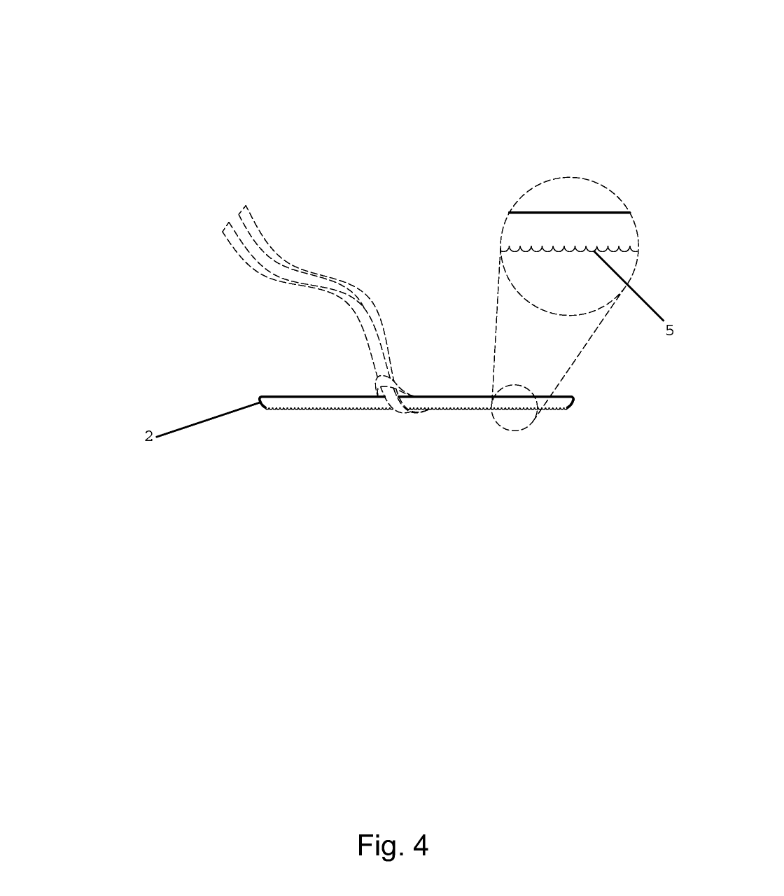

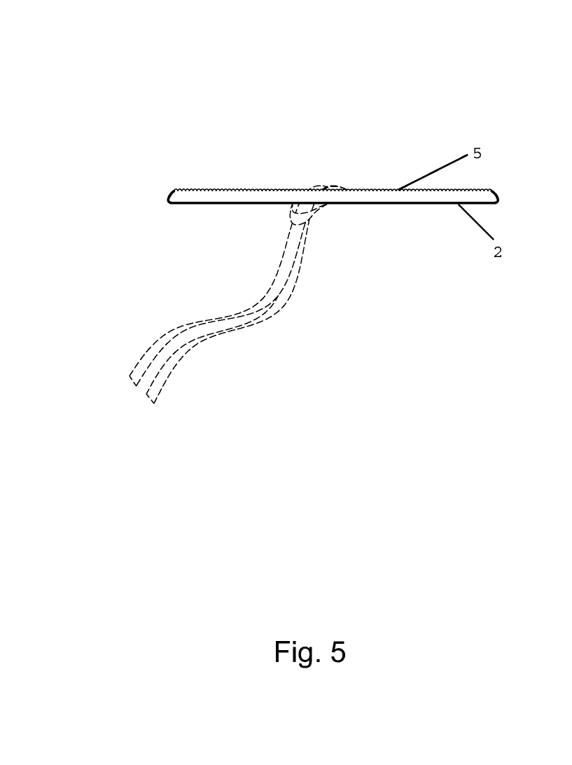

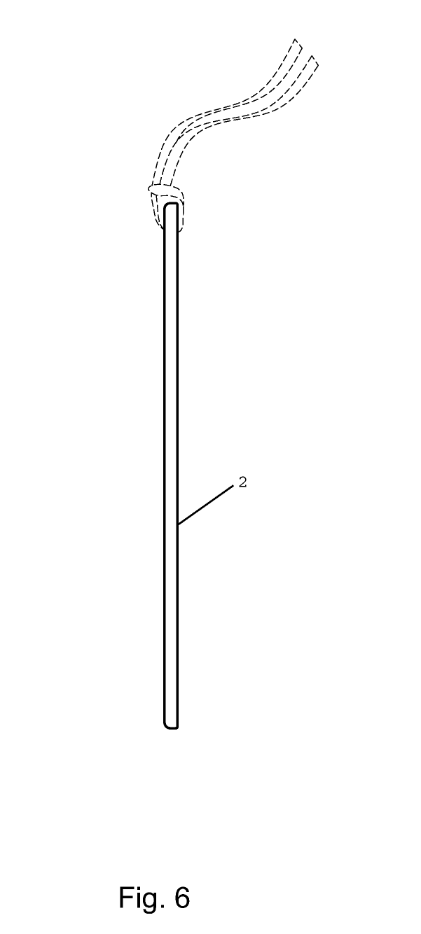

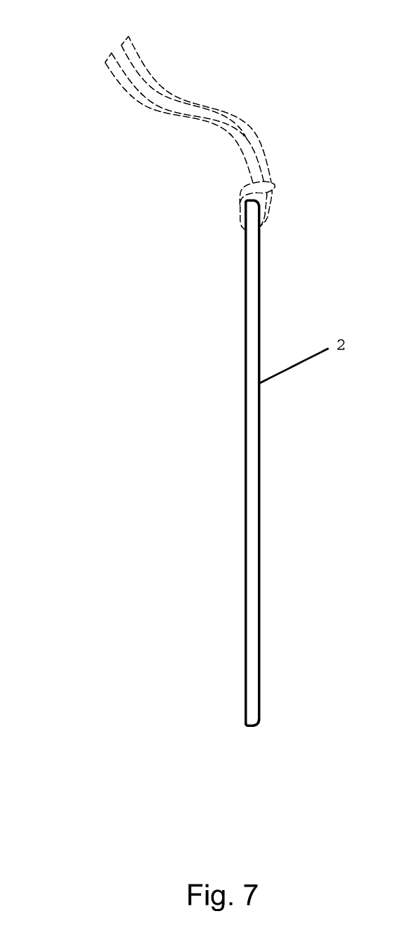

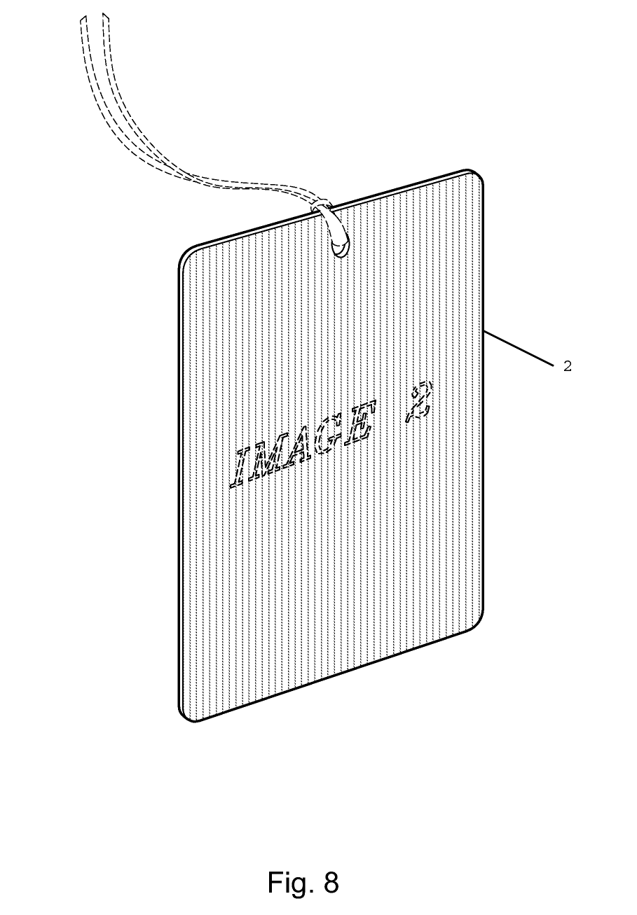

As generally shown in FIGS. 2-4, a scent sleeve (1) may be affixed to a lenticular image (2). In a preferred embodiment, a scent sleeve (1) may include a plastic scent sleeve. However, in additional embodiments a hard-plastic scent sleeve, or even a detachable scent sleeve may be used. Again, in a preferred embodiment, a scent cartridge (3) may be pre-impregnated with, for example a scented oil that will disperse when exposed to the environment. In a preferred embodiment, one or more scent cartridges (3) may be formed from an appropriate adsorbent material and further impregnated with the desired scent, perhaps through the application of one or more scented oils or other scent dispersing compounds. Naturally, such scent cartridges (3) may be any adsorbent material that can be impregnated, hold and disperse the scent. This scent cartridge (3) may be made from a polymer or paper and may be coated after it is stamped/die cut or molded. Such a scent cartridge (3) may be formed to correspond to a scent sleeve (1) affixed to the surface of a lenticular image (2) as shown in FIG. 3. In a preferred embodiment, one or more scent impregnated elements may be individually packaged, for example in a plastic or polybag to prevent the scent's dispersal into the environment. Such individually packaged scent items may further be packaged with a corresponding lenticular image (2) having a corresponding scent sleeve (1).

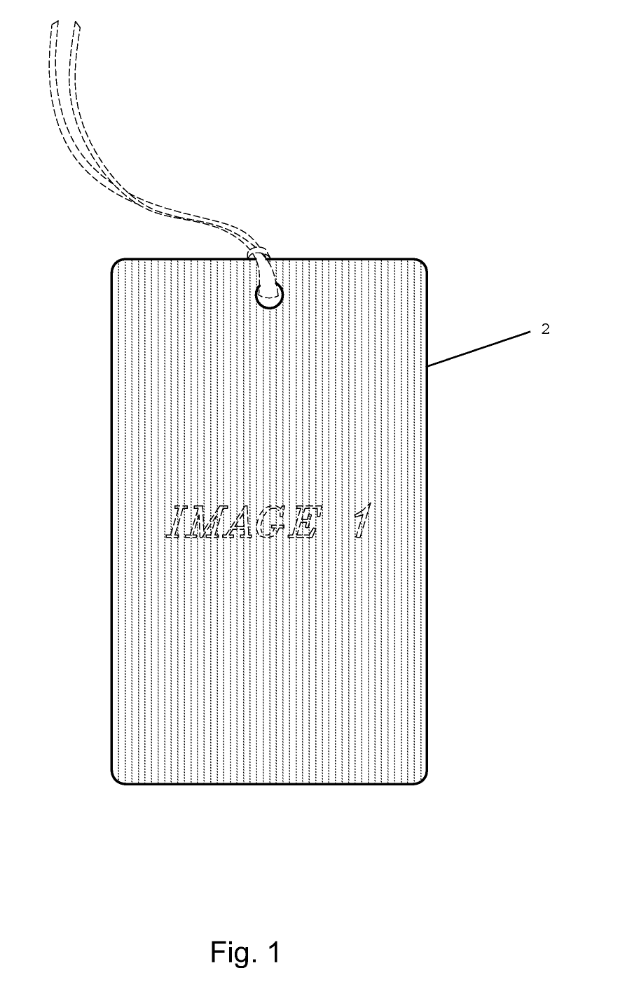

In a preferred embodiment, a user may extract one or more scent cartridges (3) and place them into a scent sleeve (1). As shown in FIG. 1, this scent sleeve (1) may be made of an impermeable material such as plastic. In this manner, the scent sleeve (1) forms a barrier between the lenticular image, plastic lenses and printing ink that would normally be destroyed by scented oils. Further, as shown in FIG. 1, the external surface of the scent sleeve (1) may have one or more perforations (4) to allow dispersion of a scented oil as they are released into the environment. Naturally, as the scented oils of one scent cartridge (3) are fully dispersed, a user may replace it with another fresh scent cartridge (3) extending the life and usefulness of the product or may re-apply a scent, perhaps in the form of a scented oil to reuse the same scent cartridge (3).

Naturally, one advantage of the invention is that a user may both enjoy a lenticular image (2) as well as the desired scent. In certain embodiments, the scent element may be personalized, such as with an image corresponding with the lenticular image (2), or with, for example another personalized identifier, such as those disclosed in U.S. Provisional Application No. 61/920,389 herein incorporated in its entirety by reference. In other embodiments, the scent may be personalized to a user's desired scent. Additionally, such scent cartridges (3) may be individually sold for additional use the lenticular image and may also display a corresponding image or other personalized image, such as a name, logo, or geographic or location identifier. Additionally, the perforations (4) integral to the scent sleeve (1) may be adjustable such that a user may increase the size and/or number of the perforations (4) to allow a controlled dispersal of the desired amount of scent into the environment. In a preferred embodiment, a scent sleeve (1) may have one or more etched perforations that may be removed based on a user's preference for scent dispersal.

In one embodiment, a scent sleeve (1) may be adapted to a souvenir, such as a lenticular magnet, cell phone case, as well as even a keychain or other souvenir or FOB type device. In an additional embodiment, such lenticular devices may be secured to a surface. For example, in a preferred embodiment, a lenticular device may be hung at a specific location through a string support, or even a hook device, while in other embodiments, one or more lenticular devices may be secured to a surface by a corresponding suction or magnet support.

Naturally, all embodiments discussed herein are merely illustrative and should not be construed to limit the scope of the inventive technology consistent with the broader inventive principles disclosed. As may be easily understood from the foregoing, the basic concepts of the present inventive technology may be embodied in a variety of ways. It generally involves systems, methods, techniques as well as devices to accomplish providing methods and apparatus for an improved scented lenticular air freshener and the like. In this application, the improved techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the statements of invention. As can be easily understood from the foregoing, the basic concepts of the present invention may be embodied in a variety of ways. It involves both techniques as well as devices to accomplish the appropriate system for providing for the improved scented lenticular air freshener and the like. In this application, the techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in method-oriented terminology, each element of the claims corresponds to a device. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting any claims. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “perforation” should be understood to encompass disclosure of the act of “perforating”—whether explicitly discussed or not—and, conversely, were there effectively disclosure of the act of “perforating”, such a disclosure should be understood to encompass disclosure of a “perforating method and/or technique, and or device” and even a “means for perforating.” Such changes and alternative terms are to be understood to be explicitly included in the description.

Any patents, publications, or other references mentioned in this application for patent, such as in the specification or an IDS are hereby incorporated herein by reference in their entirety. Any priority case(s) claimed by this application is hereby appended and hereby incorporated herein by reference in their entirety. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated herein by reference in their entirety. Finally, all references listed in the list of References To Be Incorporated By Reference In Accordance With The Patent Application or other information disclosure statement and the like filed with the application are hereby appended and hereby incorporated herein by reference in their entirety, however, as to each of the above, to the extent that such information or statements incorporated by reference might be considered inconsistent with the patenting of this/these invention(s) such statements are expressly not to be considered as made by the applicant(s).

Thus, the applicant(s) should be understood to have support to claim and make a statement of invention to at least: i) each of the system (with corresponding methods and apparatus) of providing methods and/or apparatus for the improved scented lenticular air freshener as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) the various combinations and permutations of each of the elements disclosed, xii) each potentially dependent claim or concept as a dependency on each and every one of the independent claims or concepts presented, and xiii) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. It should be understood that this application also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The inventive subject matter is to include, but certainly not be limited as, a system substantially as herein described with reference to any one or more of the Figures and Description (including the following: for example, the process according to any claims and further comprising any of the steps as shown in any Figures, separately, in any combination or permutation).

What is claimed is:

1. An improved lenticular air freshener comprising:
at least one lenticular device having a plurality of interlaced static images forming at least one single interlaced composite image;
a plurality of lenses positioned over said single interlaced composite image such that rotation of said lenticular device causes one or more portions of said single interlaced composite image to be presented to a viewer through said lenses;
at least one scent sleeve affixed to said lenticular device;
at least one perforation in said scent sleeve;
at least one scent cartridge that may be secured within said scent sleeve; and
at least one dispersible scent impregnated into said scent cartridge.

2. An improved lenticular air freshener as described in claim 1 wherein said single interlaced composite image comprises a single interlaced composite image printed onto paper and bonded to a plurality of lenses.

3. An improved lenticular air freshener as described in claim 1 wherein said single interlaced composite image comprises a single interlaced composite image printed onto a synthetic substrate and lamented to a plurality of lenses.

4. An improved lenticular air freshener as described in claim 1 wherein said lenticular device comprises a lenticular device selected from the group consisting of: a lenticular transforming print device; a lenticular animated print device; a 3-D lenticular device; and a lenticular stereoscopic device.

5. An improved lenticular air freshener as described in claim 1 wherein said scent sleeve comprises a scent sleeve selected from the group consisting of: a plastic scent sleeve; a hard-plastic scent sleeve; a detachable scent sleeve.

6. An improved lenticular air freshener as described in claim 1 wherein said perforation in said scent sleeve comprises at least one adjustable perforation in said scent sleeve.

7. An improved lenticular air freshener as described in claim 1 wherein said scent cartridge comprises a scent cartridge selected from the group consisting of: a polymer scent cartridge; a paper scent cartridge; and an adsorbent scent cartridge.

8. An improved lenticular air freshener as described in claim 1 wherein said scent cartridge comprises a scent cartridge displaying an image.

9. An improved lenticular air freshener as described in claim 8 wherein said scent cartridge displaying an image comprises a scent cartridge displaying at least one personalized image.

10. An improved lenticular air freshener as described in claim 1 wherein said single interlaced composite image comprises at least one personalized image.

11. An improved lenticular air freshener as described in claim 1 wherein said scent cartridge comprises a scent cartridge secured within a polybag.

12. An improved lenticular air freshener as described in claim 1 wherein said scent cartridge comprises a reusable a scent cartridge.

13. An improved lenticular air freshener as described in claim 1 wherein said dispersible scent comprises at least one scented oil.

14. An improved lenticular air freshener as described in claim 1 wherein said scent sleeve comprises a scent sleeve selected from the group consisting of: a scent sleeve displaying at least one personalized image; and a scent sleeve displaying at least one image.

15. An improved lenticular air freshener as described in claim 1 and further comprising a lenticular device support member.

16. An improved lenticular air freshener as described in claim 15 wherein said lenticular device support member comprises a lenticular device support member selected from the group consisting of: a string support; a hook support; a suction support; and a magnet support.

17. An improved scented lenticular magnet air freshener comprising:
at least one lenticular device having a plurality of interlaced static images forming at least one single interlaced composite image;
a plurality of lenses positioned over said single interlaced composite image such that rotation of said lenticular device causes one or more portions of said single interlaced composite image to be presented to a viewer through said lenses;
at least one magnet coupled with said lenticular device;
at least one scent sleeve affixed to said lenticular device;
at least one perforation in said scent sleeve;
at least one scent cartridge that may be secured within said scent sleeve; and
at least one dispersible scent impregnated into said scent cartridge.

18. An improved scented lenticular magnet as described in claim 1 wherein said lenticular device comprises a lenticular device selected from the group consisting of: a lenticular transforming print device; lenticular animated print device; a 3-D lenticular device; and a lenticular stereoscopic device.

19. An improved scented lenticular cell phone case comprising:
at least one lenticular cell phone case having a plurality of interlaced static images forming at least one single interlaced composite image;
a plurality of lenses positioned over said single interlaced composite image such that rotation of said lenticular device causes one or more portions of said single interlaced composite image to be presented to a viewer through said lenses;
at least one scent sleeve affixed to said lenticular device;
at least one perforation in said scent sleeve;
at least one scent cartridge that may be secured within said scent sleeve; and
at least one dispersible scent impregnated into said scent cartridge.

20. An improved scented lenticular cell phone case as described in claim 1 wherein said lenticular cell phone case comprises lenticular cell phone case selected from the group consisting of: a lenticular transforming print cell phone case; lenticular animated print cell phone case; a 3-D lenticular cell phone case; and a lenticular stereoscopic cell phone case.
